(19) United States
(12) Patent Application Publication
(10) Pub. No.: US 2020/0121175 A1
(43) Pub. Date: Apr. 23, 2020

(54) IMAGE PROCESSING DEVICE, ENDOSCOPE APPARATUS, AND OPERATING METHOD OF IMAGE PROCESSING DEVICE

(71) Applicant: OLYMPUS CORPORATION, Tokyo (JP)

(72) Inventor: Yasunori MORITA, Tokyo (JP)

(73) Assignee: OLYMPUS CORPORATION, Tokyo (JP)

(21) Appl. No.: 16/718,464

(22) Filed: Dec. 18, 2019

Related U.S. Application Data
(63) Continuation of application No. PCT/JP2017/022795, filed on Jun. 21, 2017.

(51) Int. Cl. A61B 1/06; G06T 7/00; A61B 1/04

(52) U.S. Cl. CPC A61B 1/0684; G06T 7/0012; G06T 2207/30101; G06T 2207/10068; A61B 1/04

(57) ABSTRACT

An image processing device includes a processor including hardware. The processor performs a color attenuation process on regions other than a yellow region in a captured image including a subject image to relatively enhance the visibility of the yellow region in the captured image. The processor detects a blood region that is a region of blood in the captured image based on color information of the captured image. The processor suppresses or stops an attenuation process on the blood region based on detection result of the blood region.

### CROSS REFERENCE TO RELATED APPLICATION

This application is a continuation of International Patent Application No. PCT/JP2017/022795, having an international filing date of Jun. 21, 2017, which designated the United States, the entirety of which is incorporated herein by reference.

### BACKGROUND

WO 2013/115323 discloses a method by which to separately capture reflected light in first to third wavelength bands according to absorption characteristics of carotene and hemoglobin to acquire first to third reflected light images, and display a combined image formed by combining the first to third reflected light images in different colors, thereby improving the visibility of the subject of a specific color (carotene in this case) in the body cavity.

In addition, WO 2016/151676 discloses a method by which to acquire a plurality of spectral images, calculate the amount of a separation target component using the plurality of spectral images, and perform a highlighting process on an RGB color image based on the amount of the separation target component. In the highlighting process, with a decrease in the amount of the separation target component that is the amount of the component of the subject to be increased in visibility, a luminance signal and a color difference signal are more attenuated to improve the visibility of the specific color of the subject.

As described above, there is known a method for improving the visibility of the specific color of the subject by highlighting the specific color in the body or attenuating the color with a smaller amount of component of the specific color.

### SUMMARY

According to one aspect of the invention, there is provided an image processing device comprising a processor including hardware,

the processor being configured to perform:

executing a color attenuation process on a region other than a yellow region in a captured image including a subject image to relatively enhance visibility of the yellow region in the captured image;

detecting a blood region that is a region of blood in the captured image based on color information of the captured image; and

suppressing or stopping the attenuation process on the blood region based on detection result of the blood region.

According to another aspect of the invention, there is provided an endoscope apparatus comprising an image processing device, wherein

the image processing device includes

a processor including hardware,

the processor being configured to perform:

executing a color attenuation process on a region other than a yellow region in a captured image including a subject image to relatively enhance visibility of the yellow region in the captured image;

detecting a blood region that is a region of blood in the captured image based on color information of the captured image; and

suppressing or stopping the attenuation process on the blood region based on detection result of the blood region.

According to another aspect of the invention, there is provided an operating method of an image processing device, comprising:

executing a color attenuation process on a region other than a yellow region in a captured image including a subject image to relatively enhance visibility of the yellow region in the captured image;

detecting a blood region that is a region of blood in the captured image based on color information of the captured image; and

suppressing or stopping the attenuation process on the blood region based on detection result of the blood region.

### BRIEF DESCRIPTION OF THE DRAWINGS

FIG. 1A and FIG. 1B are diagrams illustrating examples of images of inside of a body captured with an endoscope (rigid scope) during surgery.

FIG. 2 is a configuration example of an endoscope apparatus according to the present embodiment.

FIG. 3A is a diagram illustrating absorption characteristics of hemoglobin and absorption characteristics of carotene. FIG. 3B is a diagram illustrating transmittance characteristics of color filters of an image sensor. FIG. 3C is a diagram illustrating an intensity spectrum of white light.

FIG. 4 is a diagram illustrating a first detailed configuration example of an image processing section.

FIG. 5 is a diagram illustrating an operation of a blood region detection section.

FIG. 6 is a diagram illustrating an operation of a visibility enhancement section.

FIG. 7 is a diagram illustrating an operation of a visibility enhancement section.

FIG. 8 is a diagram illustrating an operation of a visibility enhancement section.

FIG. 9 is a diagram illustrating a second detailed configuration example of the image processing section.

FIG. 10 is a diagram illustrating a first modification example of the endoscope apparatus according to the present embodiment.

FIG. 11A is a diagram illustrating absorption characteristics of hemoglobin and absorption characteristics of carotene. FIG. 11B is a diagram illustrating an intensity spectrum of light emitted by a light emitting diode.

FIG. 12 is a diagram illustrating a second modification example of the endoscope apparatus according to the present embodiment.

FIG. 13 is a diagram illustrating a detailed configuration example of a filter turret.

FIG. 14A is a diagram illustrating absorption characteristics of hemoglobin and absorption characteristics of carotene. FIG. 14B is a diagram illustrating transmittance characteristics of a filter group in the filter turret.

FIG. 15 is a diagram illustrating a third modification example of the endoscope apparatus according to the present embodiment.

FIG. 16A is a diagram illustrating absorption characteristics of hemoglobin and absorption characteristics of carotene. FIG. 16B is a diagram illustrating spectral transmittance characteristics of a color separation prism 34.

FIG. 17 is a diagram illustrating a third detailed configuration example of the image processing section.

FIG. 18 is a diagram illustrating a configuration example of a surgery support system.

### DESCRIPTION OF EXEMPLARY EMBODIMENTS

The following disclosure provides many different embodiments, or examples, for implementing different features of the provided subject matter. These are, of course, merely examples and are not intended to be limiting. In addition, the disclosure may repeat reference numerals and/or letters in the various examples. This repetition is for the purpose of simplicity and clarity and does not in itself dictate a relationship between the various embodiments and/or configurations discussed. Further, when a first element is described as being “connected” or “coupled” to a second element, such description includes embodiments in which the first and second elements are directly connected or coupled to each other, and also includes embodiments in which the first and second elements are indirectly connected or coupled to each other with one or more other intervening elements in between.

Exemplary embodiments are described below. Note that the following exemplary embodiments do not in any way limit the scope of the content defined by the claims laid out herein. Note also that all of the elements described in the present embodiment should not necessarily be taken as essential elements.

For example, hereinafter, an application example of the present disclosure to a rigid scope used for surgery or the like will be described. However, the present disclosure is also applicable to a flexible scope used in an endoscope for digestive tract and the like.

1. Endoscope Apparatus and Image Processing Section

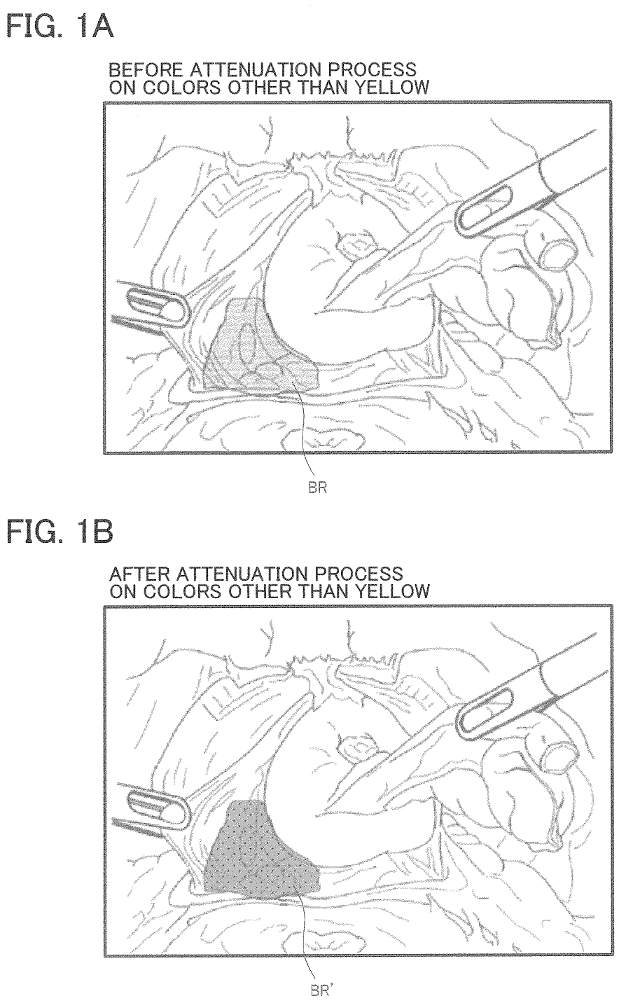

FIG. 1A illustrates an example of an image of inside of a body captured with an endoscope (rigid scope) during surgery. In such an image of inside of a body, it is difficult to directly see nerves because they are transparent. Thus, the positions of the nerves that are not directly seen are estimated by visually recognizing fat existing around the nerves (the nerves pass through the fat). The fat in the inside of a body contains carotene and appears to take on a yellow tinge by absorption characteristics (spectral characteristics) of the carotene.

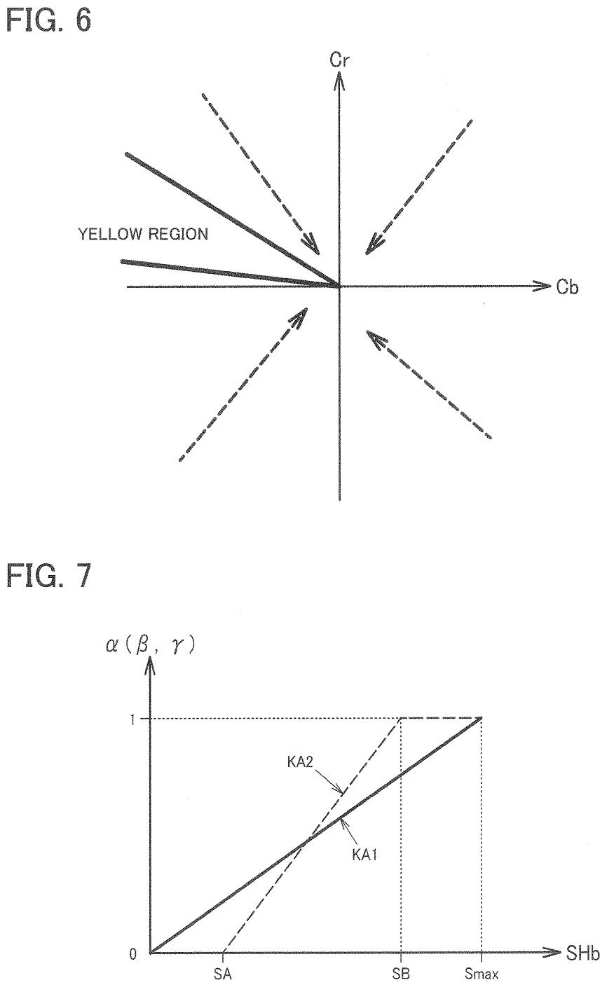

Thus, in the present embodiment, a captured image is subjected to a process of attenuating color differences in colors other than yellow (specific color) so that the visibility of the subject is relatively improved (the yellow subject is highlighted) as illustrated in FIG. 6. This makes it possible to improve the visibility of the fat through which the nerves are likely to pass.

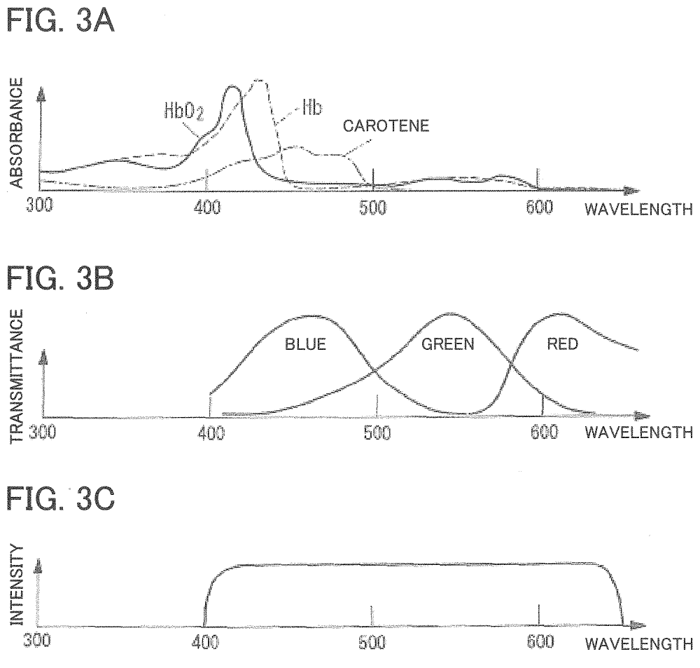

As indicated with BR in FIG. 1A, blood may exist on the subject due to bleeding or the like (or internal bleeding) during surgery. In addition, the subject has blood vessels. As a larger amount of blood exists on the subject, the blood absorbs a larger quantity of light. The wavelength of the light absorbed depends on the absorption characteristics of hemoglobin. As illustrated in FIG. 3A, the absorption characteristics of hemoglobin and the absorption characteristics of carotene are different from each other. Accordingly, as illustrated with BR′ in FIG. 1B, when the process of attenuating the colors other than yellow is performed, the color differences (chromas) in the region where the blood exists (the outflowing blood and the blood vessels) become attenuated. For example, a region where the blood accumulates may be darkened due to the blood's absorption of light. In a case where the chroma of the region becomes lower, the region appears in the image as a dark region with low chroma. Otherwise, with a decrease in chroma, the low-contrast blood vessels may further become lower in contrast.

Thus, in the present embodiment, a region where blood exists is detected from a captured image, and a display mode of a display image is controlled based on the detection result (for example, the process of attenuating the colors other than yellow is controlled). Hereinafter, an image processing device and an endoscope apparatus including the image processing device according to the present embodiment will be described.

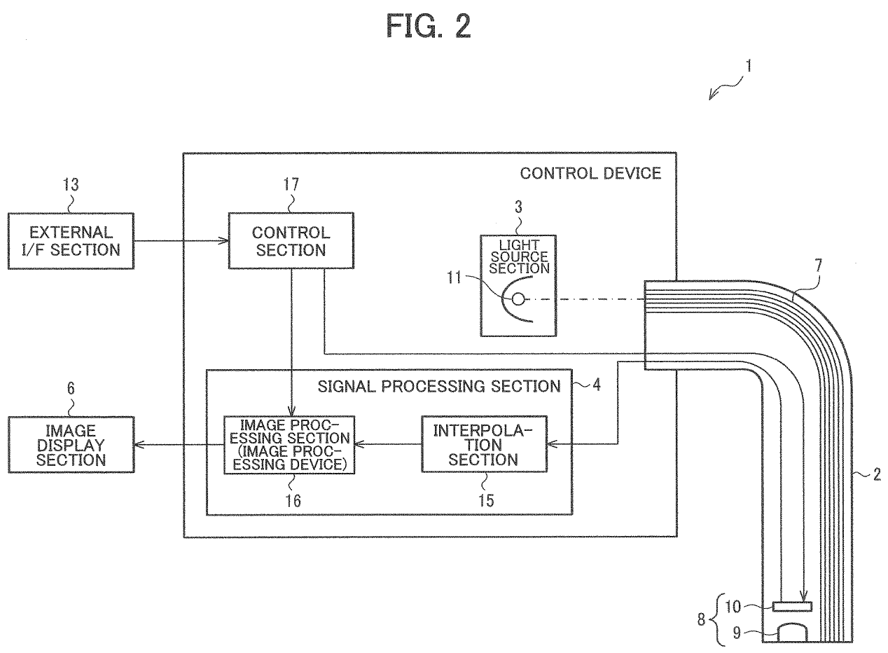

FIG. 2 illustrates a configuration example of the endoscope apparatus according to the present embodiment. An endoscope apparatus 1 (endoscope system, living body observation device) illustrated in FIG. 2 includes: an insertion section 2 (scope) to be inserted into a living body; a control device 5 (main body section) having a light source section 3 (light source device), a signal processing section 4, and a control section 17 connected to the insertion section 2; an image display section 6 (display, display device) that displays an image generated by the signal processing section 4; and an external I/F section 13 (interface).

The insertion section 2 includes an illumination optical system 7 that emits light input from the light source section 3 toward a subject and a imaging optical system 8 (imaging device, imaging section) that captures reflected light from the subject. The illumination optical system 7 is a light guide cable that is arranged along the entire longitudinal side of the insertion section 2 to guide incident light from the light source section 3 at a proximal end to a distal end.

The imaging optical system 8 includes an objective lens 9 that collects reflected light from the subject having reflected the light emitted from the illumination optical system 7 and an image sensor 10 that captures the light collected by the objective lens 9. The image sensor 10 is, e.g., a single-plate color image sensor, which is a CCD image sensor or a CMOS image sensor, for example. As illustrated in FIG. 3B, the image sensor 10 includes color filters (not illustrated) that have transmittance characteristics of respective colors of RGB (red, green, and blue).

The light source section 3 includes a xenon lamp 11 (light source) that emits white light (normal light) in a wide wavelength band. As illustrated in FIG. 3C, the xenon lamp 11 emits white light in an intensity spectrum with a wavelength band of 400 to 700 nm, for example The light source of the light source section 3 is not limited to the xenon lamp and may be any light source that can emit white light.

The signal processing section 4 includes an interpolation section 15 that processes an image signal acquired by the image sensor 10 and an image processing section 16 (image processing device) that processes the image signal processed by the interpolation section 15. The interpolation section 15 turns a color image acquired by pixels of the image sensor 10 corresponding to the individual colors (so-called Bayer array image) into a three-channel image by a publicly known demosaicing process (generating a color image with pixel values of RGB in pixels).

The control section 17 synchronizes the timing for capturing by the image sensor 10 and the timing for image processing by the image processing section 16, based on an instructive signal from the external I/F section 13.

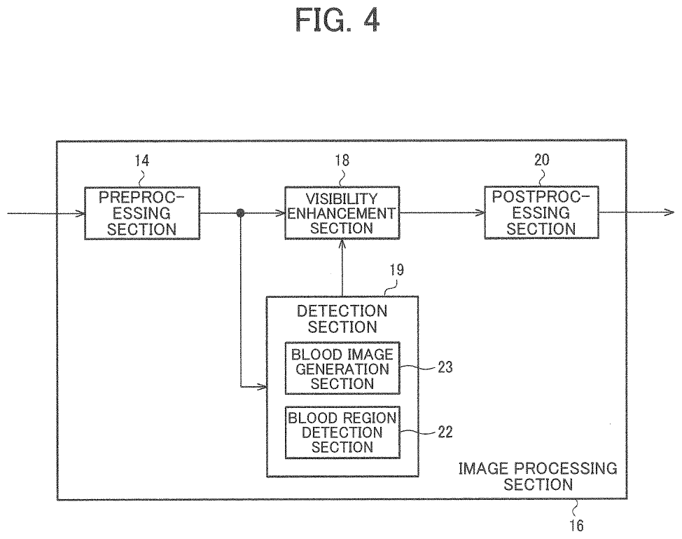

FIG. 4 illustrates a first detailed configuration example of the image processing section. The image processing section 16 includes a preprocessing section 14, a visibility enhancement section 18 (yellow enhancement section), a detection section 19 (blood detection section), and a postprocessing section 20.

Hereinafter, the case where the subject to be improved in visibility is carotene in fat will be described. As illustrated in FIG. 3A, carotene contained in biological tissue has high absorption characteristics in a region of 400 to 500 nm. In addition, hemoglobin (HbO₂, Hb) as a component of blood has high absorption characteristics in a wavelength band of 450 nm or less and a wavelength band of 500 to 600 nm. Accordingly, when white light is applied, carotene looks yellow and blood looks red. More specifically, when white light as illustrated in FIG. 3C is emitted to capture an image of the subject by the image sensor with spectral characteristics as illustrated in FIG. 3B, the pixel values of the subject containing carotene have more yellow components and the pixel values of the subject containing blood have more red components.

In the image processing section 16 illustrated in FIG. 4, using these absorption characteristics of carotene and blood, the detection section 19 detects the blood from the captured image, and the visibility enhancement section 18 performs a process of improving the visibility of the color of carotene (yellow in a broad sense). Then, the visibility enhancement section 18 controls the process of improving the visibility using the detection result of the blood. Hereinafter, the parts of the image processing section 16 will be described in detail.

The preprocessing section 14 performs an optical black (OB) clamp process, a gain correction process, and a white balance (WB) correction process on the three-channel image signals input from the interpolation section 15, using an OB clamp value, a gain correction value, and a WB coefficient value saved in advance in the control section 17. Hereinafter, the image processed and output by the preprocessing section 14 (RGB color image) will be called captured image.

The detection section 19 includes a blood image generation section 23 that generates a blood image based on the captured image from the preprocessing section 14 and a blood region detection section 22 (outflowing blood region detection section) that detects a blood region (outflowing blood region in a narrow sense) based on the blood image.

As described above, the image signals after the preprocessing include three types (three channels) of image signals of blue, green, and red. The blood image generation section 23 generates one channel of image signal from the two types (two channels) of image signals of green and red and forms the blood image from the image signal. In the blood image, the pixels with a larger amount of hemoglobin contained in the subject have higher pixel values (signal values). For example, the blood image generation section 23 generates the blood image by determining the differences between the pixel values of red and the pixel values of green in each pixel. Otherwise, the blood image generation section 23 generates the blood image by dividing the pixel value of red by the pixel value of green in each pixel.

In the example described above, the blood image is generated from the two channels of signals. However, the present disclosure is not limited to this, and the blood image may be generated by calculating luminance (Y) and color differences (Cr, Cb) from the three channels of RGB signals, for example. In that case, the blood image generation section 23 generates the blood image from the color difference signal such that the region where the chroma of red is sufficiently high or the region where the luminance signal is low to some degree is the region where blood exists. For example, the blood image generation section 23 determines an index value corresponding to the chroma of red for each pixel based on the color difference signal, and generates the blood image from the index values. Otherwise, the blood image generation section 23 determines the index value that becomes larger as the luminance signal is lower for each pixel based on the luminance signal, and generates the blood image from the index values.

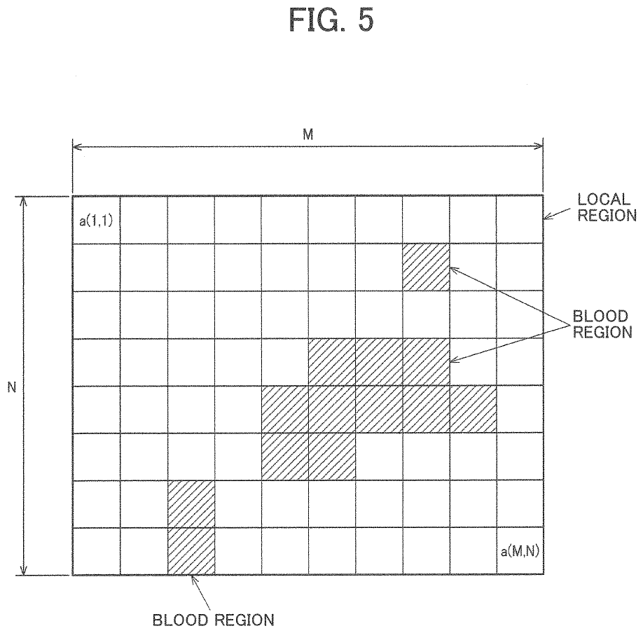

The blood region detection section 22 sets a plurality of local regions (divided regions, blocks) in the blood image. For example, the blood region detection section 22 divides the blood image into a plurality of rectangular areas, and sets the divided rectangular areas as local regions. The size of rectangular areas can be set as appropriate but one local region is set to 16×16 pixels, for example. For example, as illustrated in FIG. 5, the blood image is divided into M×N local regions, and the coordinates of each local region are represented by (m, n) where m is an integer of 1 or more and M or less, and n is an integer of 1 or more and N or less. The local region in the coordinates (m, n) is indicated as a(m, n). Referring to FIG. 5, the coordinates of the local region located at the upper left of the image are (1, 1), the right direction is set to a forward direction of m, and the downward direction is set to a forward direction of n.

The local regions are not necessarily rectangular. It is obvious that the blood image can be divided into any polygonal shape and the divided regions can be set as local regions. In addition, the local regions may be appropriately settable in response to the operator's instruction. In the present embodiment, a region formed from a group of a plurality of adjacent pixels is set as one local region for the sake of reducing the amount of calculation later and removing noise. However, one pixel can be set as one local region. In this case, the following process is the same.

The blood region detection section 22 sets the blood region where blood exists in the blood image. That is, the blood region detection section 22 sets the region with a large amount of hemoglobin as the blood region. For example, the blood region detection section 22 performs a threshold process on all the local regions to extract local regions with sufficiently large values of blood image signals, performs an integration process on adjacent local regions, and sets the resultant regions as the blood region. In the threshold process, for example, the blood region detection section 22 compares values obtained by averaging the pixel values in the local regions with a given threshold, and extracts local regions with the averaged values larger than the given threshold. The blood region detection section 22 calculates the positions of all the pixels included in the blood region from coordinates a(m, n) of the local regions included in the blood region and information about the pixels included in the local regions, and outputs the calculated information to the visibility enhancement section 18 as blood region information indicating the blood region.

The visibility enhancement section 18 subjects the captured image from the preprocessing section 14 to a process of decreasing the chromas of the regions other than the yellow region in a color difference space. Specifically, the visibility enhancement section 18 converts the image signals of RGB pixels in the captured image into a YCbCr signal of luminance color difference. The conversion equations are the following (1) to (3):

Y=0.2126×R+0.7152×G+0.0722×B (1)

Cb=−0.114572×R−0.385428×G+0.5×B (2)

Cr=0.5×R−0.454153×G−0.045847×B (3)

Next, as illustrated in FIG. 6, the visibility enhancement section 18 attenuates the color differences in the regions other than the yellow region in the color difference space. The range of yellow in the color difference space is defined by the range of angles with reference to a Cb axis, for example. Thus, the color difference signals are not attenuated for the pixels in which the color difference signals fall within the range of angles.

Specifically, as shown in the following equations (4) to (6), the visibility enhancement section 18 controls the amount of attenuation according to the signal value of the blood image in the blood region detected by the blood region detection section 22. In the regions other than the blood region (excluding the yellow region), coefficients α, β, and γ are fixed to values smaller than 1, for example. Otherwise, in the regions other than the blood region (excluding the yellow region), the amount of attenuation may be controlled by the following equations (4) to (6):

Y′=α(SHb)×Y (4)

Cb′=β(SHb)×Cb (5)

Cr′=γ(SHb)×Cr (6)

where SHb represents the signal value (pixel value) of the blood image. As illustrated in FIG. 7, α(SHb), β(SHb), and γ(SHb) are coefficients that vary depending on the signal value SHb of the blood image, which take a value of 0 or more and 1 or less. For example, as illustrated with KA1 in FIG. 7, the coefficients are proportional to the signal value SHb. Otherwise, as illustrated with KA2, the coefficient may be 0 when the signal value SHb is equal to or less than SA, the coefficient may be proportional to the signal value SHb when the signal value SHb is larger than SA and equal to or smaller than SB, and the coefficient may be 1 when the signal value SHb is larger than SB. The relationship 0<SA<SB<Smax holds where Smax represents the largest value of the signal value SHb. FIG. 7 illustrates a case where the coefficient changes linearly with respect to the signal value SHb. However, the coefficient may change in a curve with respect to the signal value SHb. For example, the coefficient may change in a curve that projects above KA1 or under KA1. The coefficients α(SHb), β(SHb), and γ(SHb) may be coefficients that change in the same manner with respect to the signal value SHb or may be coefficients that changes in different manners.

According to the foregoing equations (4) to (6), the coefficients come close to 1 in the region where blood exists, and thus the amount of attenuation becomes small. That is, the pixels with larger signal values in the blood image are less likely to be attenuated in color (color difference). Otherwise, in the blood region detected by the blood region detection section 22, the amount of attenuation is smaller than outside the blood region, and thus the colors (color differences) are unlikely to be attenuated.

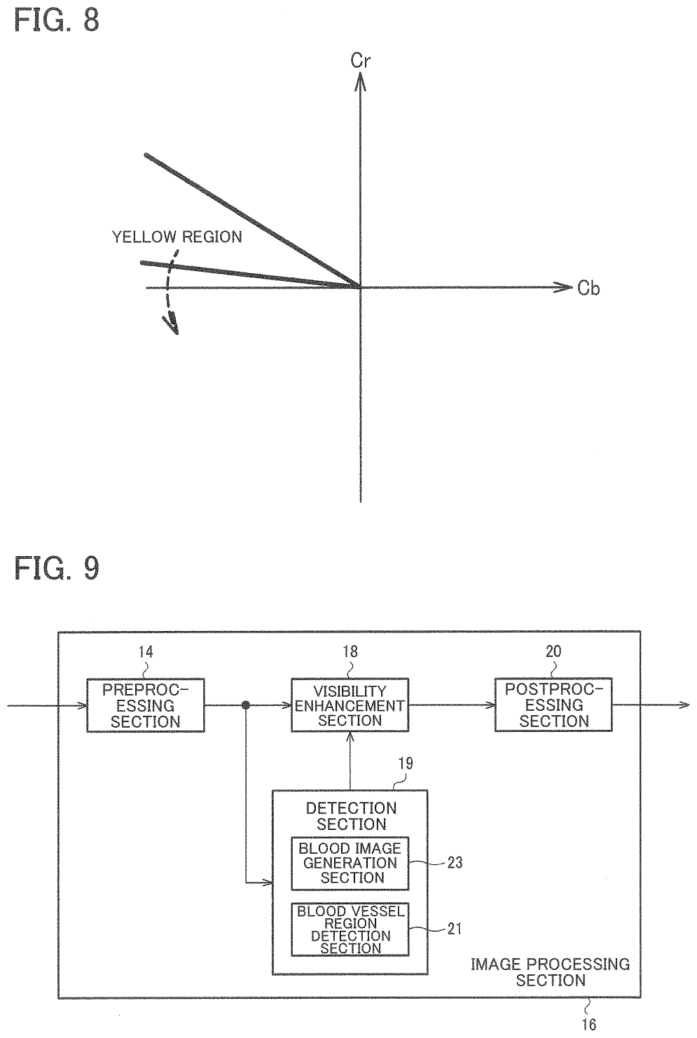

Further, as illustrated in FIG. 8, the yellow region may be rotated toward green in the color difference space. This makes it possible to enhance the contrast between the yellow region and the blood region. As described above, the color of yellow is defined by the range of angles with respect to the Cb axis. The color difference signals belonging to the angle range of yellow is rotated counterclockwise at a predetermined angle in the color difference space, thereby achieving the rotation toward green.

The visibility enhancement section 18 converts the attenuated YCbCr signal into RGB signals by the equations (7) to (9) shown below. The visibility enhancement section 18 outputs the converted RGB signals (color image) to the postprocessing section 20.

R=Y′+1.5748×Cr′ (7)

G=Y′−0.187324×Cb′−0.468124×Cr′ (8)

B=Y′+1.8556×Cb′ (9)

In the example described above, the color difference signals and the luminance signals in the regions other than the yellow region are attenuated. Alternatively, only the color difference signals in the regions other than the yellow region may be attenuated. In this case, the foregoing equation (4) is not executed, and Y′=Y in the foregoing equations (7) to (9).

In the example described above, the process of attenuating the colors other than yellow is suppressed in the blood region. However, the control method of the process of attenuating the colors other than yellow is not limited to this. For example, when the ratio of the blood region to the image exceeds a specific ratio (that is, the number of pixels in the blood region/the number of all the pixels exceeds a threshold), the process of attenuating the colors other than yellow may be suppressed in the entire image.

The postprocessing section 20 performs postprocessing such as a grayscale transmission process, a color process, and a contour highlighting process on the image from the visibility enhancement section 18 (the image in which the colors other than yellow are attenuated) using a grayscale transformation coefficient, color conversion coefficient, and contour highlighting coefficient saved in the control section 17, thereby generating a color image to be displayed on the image display section 6.

According to the foregoing embodiment, the image processing device (the image processing section 16) includes the image acquisition section (for example, the preprocessing section 14) and the visibility enhancement section 18. The image acquisition section acquires a captured image including a subject image obtained by applying illumination light from the light source section 3 to the subject. Then, as described above with reference to FIG. 6 and others, the visibility enhancement section 18 performs the color attenuation process on the regions other than the yellow region in the captured image to relatively enhance the visibility of the yellow region in the captured image (perform yellow enhancement).

This makes it possible to attenuate the chroma of tissue having the colors other than yellow of the subject seen in the captured image as compared to tissue in yellow (for example, fat containing carotene). As a result, the tissue in yellow is highlighted so that the visibility of the tissue in yellow can be enhanced relative to the tissue in the colors other than yellow. In addition, the attenuation process is performed using the captured image (for example, RGB color image) acquired by the image acquisition section, which simplifies the configuration and the processes as compared to a case where a plurality of spectral images are prepared and the attention process is performed using the plurality of spectral images.

In this case, the yellow here refers to colors that belong to a predetermined region corresponding to yellow in the color space. For example, the range of angles with reference to the Cb axis centered on a point of origin in a CbCr plane in a YCbCr space constitutes colors belonging to a predetermined angle range. Otherwise, the yellow refers to colors that belong to a predetermined angle range in a hue (H) plane in an HSV space. In addition, the yellow refers to colors between red and green in the color space, which exist in the counterclockwise direction of red and exist in the clockwise direction of green in the CbCr plane, for example. However, the yellow is not limited to the foregoing definition and may be defined by spectral characteristics of a yellow substance (for example, carotene, bilirubin, stercobilin, or the like) or a region occupied by that substance in the color space. The colors other than yellow refer to colors that do not belong to a predetermined region corresponding to yellow (but belong to the regions other than the predetermined region) in the color space, for example.

The color attenuation process is a process of decreasing the chroma of colors. For example, the color attenuation process is a process of attenuating the color difference signals (Cb signal and Cr signal) in the YCbCr space as illustrated in FIG. 6. Otherwise, the color attenuation process is a process of attenuating a chroma signal (S signal) in the HSV space. The color space used in the attenuation process is not limited to the YCbCr space or the HSV space.

In the present embodiment, the image processing device (the image processing section 16) includes the detection section 19 that detects the blood region as a region of blood in the captured image, based on color information of the captured image. The visibility enhancement section 18 suppresses or stops the attenuation process on the blood region based on the result of detection by the detection section 19.

As described above with reference to FIG. 3A, the absorption characteristics of hemoglobin as a component of blood and the absorption characteristics of a yellow substance such as carotene are different from each other. Thus, as described above with reference to FIG. 1B, when the color attenuation process is performed on the regions other than the yellow region, the chroma of the blood region may decrease. In this respect, in the present embodiment, the color attenuation process on the regions other than the yellow region is suppressed or stopped in the blood region. This suppresses or prevents the chroma of color in the blood region from becoming lower.

Here, the blood region is a region where it is estimated that blood exists in the captured image. Specifically, the blood region is a region with the spectral characteristics (colors) of hemoglobin (HbO₂, HbO). As described above with reference to FIG. 5, for example, the blood region is determined for each local region. This corresponds to detecting the region of blood that has a specific degree of spread (at least a local region). However, the blood region is not limited to this and may be (or include) a blood vessel region as described later with reference to FIG. 9, for example. That is, the blood region as a detection target may exist in any place of the subject that can be detected from the image and may have any shape or area. For example, the blood region can be assumed as a blood vessel (blood in the blood vessel), a region with a large number of blood vessels (for example, capillary blood vessels), blood that flows outside the blood vessel and accumulates on the surface of the subject (tissue, treatment tool, or the like), blood that flows outside the blood vessel (internal bleeding) and accumulates in the tissue, or the like.

The color information in the captured image refers to information that indicates the colors of pixels or regions of the captured image (for example, the local regions as illustrated in FIG. 5). The color information may be acquired from an image obtained by subjecting the captured image to a filtering process (an image based on the captured image), for example. The color information is a signal that is obtained by performing a calculation (for example, subtraction or division) between channels on pixel values or signal values in a region (for example, the average value of pixel values in the region), for example. Otherwise, the color information may be a pixel value or a component of a signal value in a region (channel signal). Otherwise, the color information may be a signal value obtained by converting a pixel value or signal value in a region into a signal value in a given color space. For example, the color information may be a Cb signal and a Cr signal in the YCbCr space or may be a hue (H) signal or a chroma (S) signal in the HSV space.

In the present embodiment, the detection section 19 includes the blood region detection section 22 that detects the blood region based on at least one of the color information and brightness information of the captured image. The visibility enhancement section 18 suppresses or stops the attenuation process on the blood region based on the result of detection by the blood region detection section 22. The suppression of the attenuation process means that the amount of attenuation is larger than zero (for example, the coefficients β and γ in the foregoing equations (5) and (6) are smaller than 1). The stoppage of the attenuation process means that the attenuation process is not performed or the amount of attenuation is zero (for example, the coefficients β and γ in the foregoing equations (5) and (6) are 1).

The blood accumulating on the surface of the subject becomes dark due to light absorption (for example, the blood is captured in a darker color as the width of the accumulating blood is larger). Thus, using the brightness information of the captured image makes it possible to detect the blood accumulating on the surface of the subject, thereby suppressing or preventing a decrease in the chroma of the accumulating blood.

The brightness information of the captured image here refers to information that indicates the brightness of a pixel or region (for example, the local region as illustrated in FIG. 5) of the captured image. The brightness information may be acquired from an image obtained by subjecting the captured image to a filtering process (an image based on the captured image), for example. The brightness information may be a pixel value or a component of a signal value in a region (channel signal, for example, a G signal in an RGB image), for example. Otherwise, the color information may be a signal value obtained by converting a pixel value or signal value in a region into a signal value in a given color space. For example, the brightness information may be a luminance (Y) signal in the YCbCr space or may be a brightness (V) signal in the HSV space.

In the present embodiment, the blood region detection section 22 divides the captured image into a plurality of local regions (for example, the local regions illustrated in FIG. 5), and determines whether each of the plurality of local regions is the blood region based on at least one of the color information and brightness information of the local region.

This makes it possible to determine whether each local region of the captured image is the blood region. For example, it is possible to set the region obtained by combining adjacent ones of the local regions that have been determined to be blood regions as final blood region. Determining whether the local region is the blood region makes it possible to decrease the influence of noise, thereby improving the accuracy of determination on the blood region.

In the present embodiment, based on the captured image, the visibility enhancement section 18 performs the color attenuation process on the regions other than the yellow region in the captured image. Specifically, the visibility enhancement section 18 determines the amount of attenuation (calculates the attenuation coefficient) based on the color information (color information of pixels or regions) of the captured image, and performs the color attenuation process on the regions other than the yellow region by the amount of attenuation.

Accordingly, the attenuation process is controlled (the amount of attenuation is controlled) based on the captured image. This makes it possible to simplify the configuration and the process as compared to a case where a plurality of spectral images are captured and the attenuation process is controlled based on the plurality of spectral images, for example.

In the present embodiment, the visibility enhancement section 18 performs the attenuation process by determining a color signal corresponding to the blood for the pixel or region of the captured image and multiplying the color signals in the regions other than the yellow region by the coefficient that changes in value according to the signal value of the color signal. Specifically, when the color signal corresponding to the blood is a color signal that has a signal value becoming larger in the region where the blood exists, the color signals in the regions other than the yellow region are multiplied by the coefficient that becomes larger (approaches 1) with an increase in the signal value.

For example, according to the foregoing equations (5) and (6), the color signal corresponding to the blood has a signal value SHb that is a difference value or a division value between R signal and G signal, the coefficients are β(SHb) and γ(SHb), and the color signals to be multiplied by the coefficients are color difference signals (Cb signal and Cr signal). The signal corresponding to the blood is not limited to this and may be a color signal in a given color space, for example. In addition, the color signal to be multiplied by the coefficient is not limited to the color difference signal and may be a chroma (S) signal in the HSV space or may be a component of RGB (channel signal).

This makes it possible to increase the value of the coefficient as there is a higher possibility of the existence of blood (for example, as the signal value of the color signal corresponding to the blood is larger). Multiplying the color signals in the regions other than the yellow region by the coefficient makes it possible to suppress the attenuation amount of colors as there is a higher possibility of the existence of the blood.

In the present embodiment, the visibility enhancement section 18 performs the color conversion process on the pixel values of pixels in the yellow region so as to rotate toward green in the color space.

For example, the color conversion process is a process of converting a color so as to rotate counterclockwise in the CbCr plane of the YCbCr space. Otherwise, the color conversion process is a process of converting a color so as to rotate counterclockwise in the hue (H) plane of the HSV space. For example, the visibility enhancement section 18 perform rotational conversion at an angle smaller than the angular difference between yellow and green in the CbCr plane or the hue plane.

This converts the yellow region in the captured image so as to come closer to green. Since the color of blood is red and its complementary color is green, bringing the yellow region closer to green improves the color contrast between the blood region and the yellow region, thereby further enhancing the visibility of the yellow region.

In the present embodiment, the color of the yellow region is the color of carotene, bilirubin, or stercobilin.

Carotene is a substance contained in fat, cancer, and others, for example Bilirubin is a substance contained in bile and others. Stercobilin is a substance contained in stool, urine, and others.

This makes it possible to detect the region where the existence of carotene, bilirubin, or stercobilin is estimated as the yellow region, and to perform the attenuation process on the colors other than the color of the yellow region. Accordingly, it is possible to relatively improve the visibility of the region where there exists fat, cancer, bile, stool, urine, or the like in the captured image.

The image processing device according to the present embodiment may be configured as described below. That is, the image processing device includes a memory that stores information (for example, programs and various types of data) and a processor that operates based on the information stored in the memory (a processor including hardware). The processor performs an image acquisition process of acquiring a captured image including a subject image obtained by applying illumination light from a light source section 3 to a subject and a visibility enhancement process of relatively enhancing the visibility of a yellow region in the captured image by performing a color attenuation process on regions other than a yellow region in the captured image.

For example, the processor may have functions of its sections each implemented by individual hardware, or may have the functions of its sections each implemented by integrated hardware. For example, the processor may include hardware, and the hardware may include at least one of a circuit that processes a digital signal and a circuit that processes an analog signal. For example, the processor may include one or more circuit devices (e.g., an integrated circuit (IC)) mounted on a circuit board, or one or more circuit elements (e.g., a resistor or a capacitor). The processor may be a central processing unit (CPU), for example. Note that the processor is not limited to the CPU, and various other processors such as a graphics processing unit (GPU) and a digital signal processor (DSP) may also be used. Alternatively, the processor may be a hardware circuit by ASIC. The processor may include, e.g., an amplifier circuit or a filter circuit that processes an analog signal. The memory may be a semiconductor memory (e.g., SRAM or DRAM), or may be a register. The memory may be a magnetic storage device such as a hard disk drive (HDD), or may be an optical storage device such as an optical disc device. For example, the memory stores computer-readable instructions. When the instructions are executed by the processor, the functions of components of the image processing device are implemented. The instruction described herein may be an instruction set that is included in a program, or may be an instruction that instructs the hardware circuit included in the processor to operate.

For example, operations according to the present embodiment are implemented as follows. The image captured by an image sensor 10 is processed by a preprocessing section 14 and is stored as a captured image in the memory. The processor reads the captured image from the memory, performs the attenuation process on the captured image, and stores the image having undergone the attenuation process in the memory.

The components of the image processing device according to the present embodiment may be implemented as modules of programs that run on the processor. For example, the image acquisition section is implemented as an image acquisition module that acquires a captured image including a subject image obtained by applying illumination light from the light source section 3 to a subject. A visibility enhancement section 18 is implemented as a visibility enhancement module that performs the color attenuation process on the regions other than the yellow region in the captured image to relatively enhance the visibility of the yellow region in the captured image.

2. Second Detailed Configuration Example of the Image Processing Section

FIG. 9 illustrates a second detailed configuration example of the image processing section. Referring to FIG. 9, a detection section 19 includes a blood image generation section 23 and a blood vessel region detection section 21. The configuration of an endoscope apparatus is the same as illustrated in FIG. 2. Hereinafter, the already described components will be given the same reference signs and descriptions thereof will be omitted as appropriate.

The blood vessel region detection section 21 detects a blood vessel region based on structural information of a blood vessel and a blood image. The method of generating the blood image by the blood image generation section 23 is the same as in the first detailed configuration example. The structural information of the blood vessel is detected based on a captured image from the preprocessing section 14. Specifically, the blood vessel region detection section 21 performs a direction smoothing process (noise suppression) and a high-pass filter process on a B channel (a channel with a high absorption rate of hemoglobin) of pixel values (image signals). In the direction smoothing process, the blood vessel region detection section 21 determines an edge direction with respect to the captured image. The edge direction is determined as any of horizontal direction, vertical direction, and oblique direction, for example. Next, the blood vessel region detection section 21 performs the smoothing process on the detected edge direction. The smoothing process is a process of averaging pixel values of pixels arrayed in the edge direction, for example. The blood vessel region detection section 21 performs the high-pass filter process on the image having undergone the smoothing process, thereby extracting the structural information of the blood vessel. The region in which the extracted structural information and the pixel value of the blood image are both at high levels is set as the blood vessel region. For example, the pixels in which the signal value of the structural information is larger than a first given threshold and the pixel value of the blood image is larger than a second given threshold are determined as the pixels in the blood vessel region. The blood vessel region detection section 21 outputs the information of the detected blood vessel region (the coordinates of the pixels belonging to the blood vessel region) to the visibility enhancement section 18.

The visibility enhancement section 18 controls the amount of attenuation according to the signal value of the blood image in the blood vessel region detected by the blood vessel region detection section 21. The method for controlling the amount of attenuation are the same as in the first detailed configuration example.

According to the embodiment described above, the detection section 19 includes the blood vessel region detection section 21 that detects the blood vessel region as the region of the blood vessel in the captured image based on the color information and structural information of the captured image. The visibility enhancement section 18 suppresses or stops the attenuation process on the blood vessel region based on the result of detection by the blood vessel region detection section 21.

Since a blood vessel is within tissue, the image of the blood vessel may be low in contrast depending on its thickness, depth and position in the tissue. When the color attenuation process is performed on the regions other than the yellow region, the low contrast of the blood vessel may further become lower. In this respect, according to the present embodiment, the attenuation process on the blood vessel region can be suppressed or stopped, which makes it possible to suppress or prevent a decrease in the contrast of the blood vessel region.

The structural information of the captured image here refers to extracted information on the structure of the blood vessel. For example, the structural information refers to the edge quantity of the image. The edge quantity refers to an edge quantity extracted by performing the high-pass filter process or the bandpass filter process on the image, for example. The blood vessel region refers to a region where it is estimated that a blood vessel exists in the captured image. Specifically, the blood vessel region is a region that has spectral characteristics (colors) of hemoglobin (HbO₂, HbO) and structural information (for example, edge quantity). As described above, the blood vessel region is a kind of blood region.

In the present embodiment, the visibility enhancement section 18 may enhance the structure of the blood vessel region in the captured image based on the result of detection by the blood vessel region detection section 21, and perform the attenuation process on the captured image after enhancement.

For example, the visibility enhancement section 18 may perform the structural enhancement and the attenuation process on the blood vessel region without suppressing or stopping the attenuation process on the blood region (blood vessel region). Alternatively, the visibility enhancement section 18 may suppress or stop the attenuation process on the blood region (blood vessel region) and perform the structural enhancement and attenuation processes on the blood vessel region.

Here, the process of enhancing the structure of the blood vessel region can be implemented by adding the edge quantity (edge image) extracted from an image to the captured image, for example. The structural enhancement is not limited to this.

Accordingly, the contrast of the blood vessel can be improved by the structural enhancement, and the color attenuation process is performed on the regions other than the yellow region in the blood vessel region improved in contrast. This makes it possible to suppress or prevent a decrease in the contrast of the blood vessel region.

3. Modifications

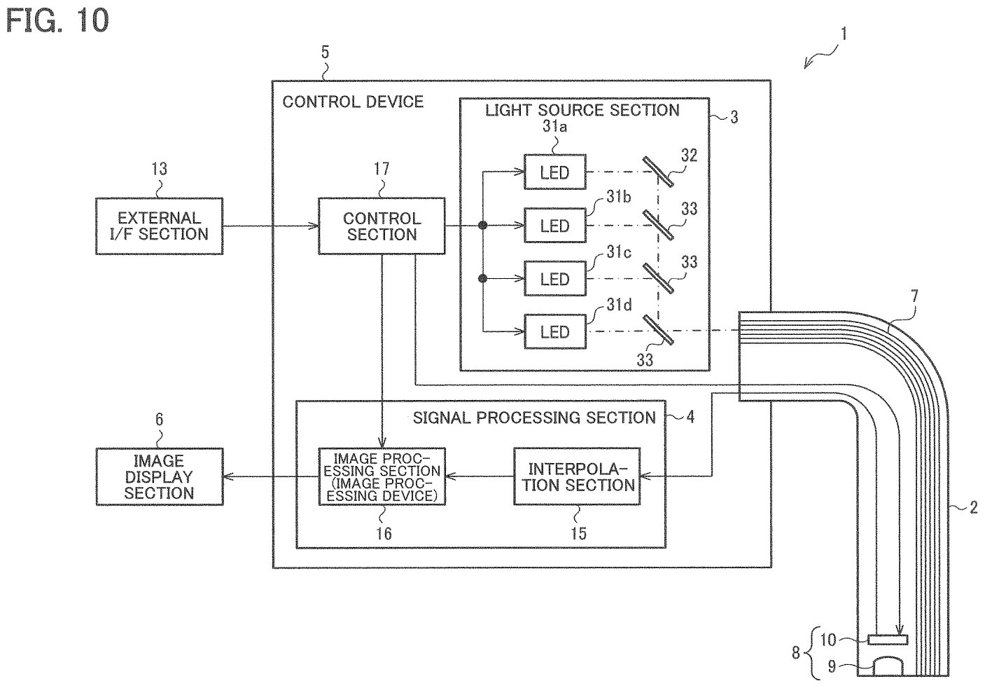

FIG. 10 illustrates a first modification of the endoscope apparatus according to the present embodiment. Referring to FIG. 10, a light source section 3 includes a plurality of light emitting diodes 31a, 31b, 31c, and 31d (LEDs) that emit light in different wavelength bands, a mirror 32, and three dichroic mirrors 33.

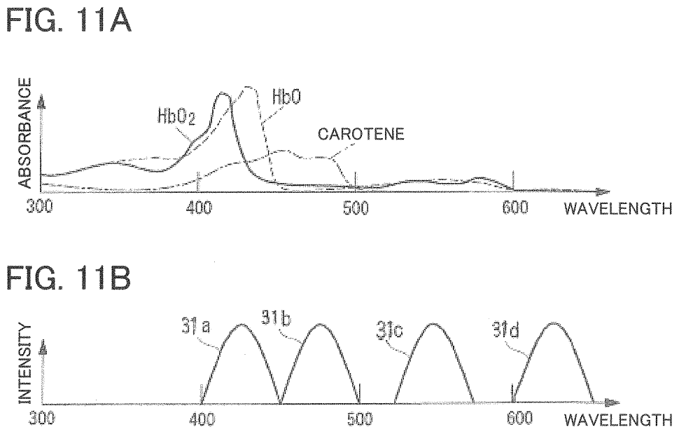

As illustrated in FIG. 11B, the light emitting diodes 31a, 31b, 31c, and 31d emit light in the wavelength bands of 400 to 450 nm, 450 to 500 nm, 520 to 570 nm, and 600 to 650 nm. For example, as illustrated in FIG. 11A and FIG. 11B, the wavelength band of the light emitting diode 31a is a wavelength band in which the absorbances of hemoglobin and carotene are both high. The wavelength band of the light emitting diode 31b is a wavelength band in which the absorbance of hemoglobin is low and the absorbance of carotene is high. The wavelength band of the light emitting diode 31c is a wavelength band in which the absorbances of hemoglobin and carotene are both low. The wavelength band of the light emitting diode 31d is a wavelength band in which the absorbances of hemoglobin and carotene are both close to zero. These four wavelength bands almost cover the wavelength band of white light (400 to 700 nm).

The light from the light emitting diodes 31a, 31b, 31c, and 31d enters an illumination optical system 7 (light guide cable) by means of the mirror 32 and the three dichroic mirrors 33. The light emitting diodes 31a, 31b, 31c, and 31d emit light at the same time such that white light is applied to the subject. An image sensor 10 is a single-plate color image sensor, for example. The wavelength bands of 400 to 500 nm of the light emitting diodes 31a and 31b correspond to the wavelength band of blue, the wavelength band 520 to 570 nm of the light emitting diode 31c corresponds to the wavelength band of green, and the wavelength band 600 to 650 nm of the light emitting diode 31d corresponds to the wavelength band of red.

The configurations of the light emitting diodes and their wavelength bands are not limited to the foregoing ones. That is, the light source section 3 is merely required to include one or more light emitting diodes such that the one or more light emitting diodes emit light to generate white light. The wavelength bands of the light emitting diodes may be randomly set as long as the light emission from the one or more light emitting diodes covers the wavelength band of white light as a whole. For example, the light emission from the one or more light emitting diodes only needs to cover the wavelength bands of red, green, and blue.

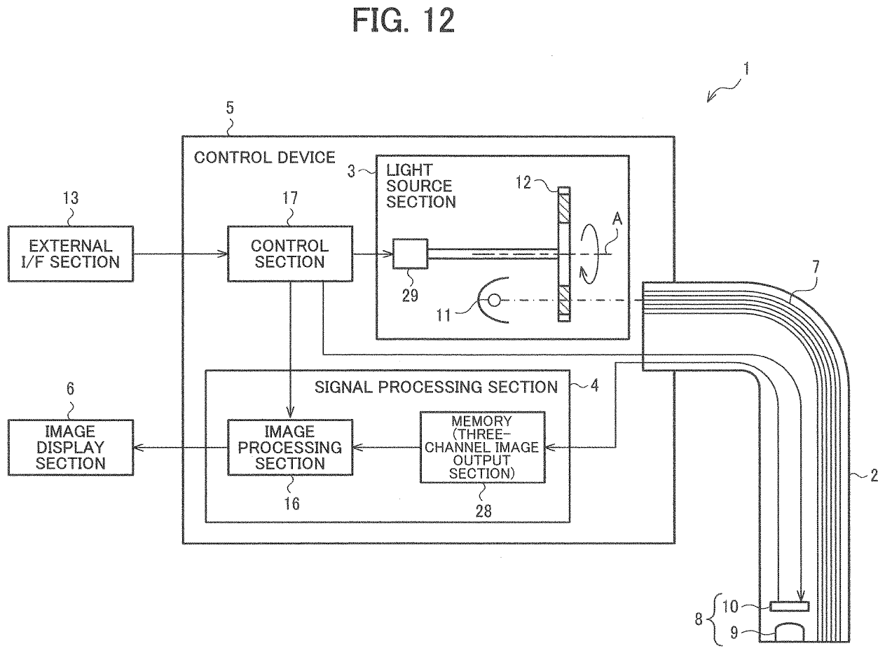

FIG. 12 illustrates a second modification of the endoscope apparatus according to the present embodiment. Referring to FIG. 12, a light source section 3 includes a filter turret 12, a motor 29 that rotates the filter turret 12, and a xenon lamp 11. A signal processing section 4 includes a memory 28 and an image processing section 16. An image sensor 27 is a monochrome image sensor.

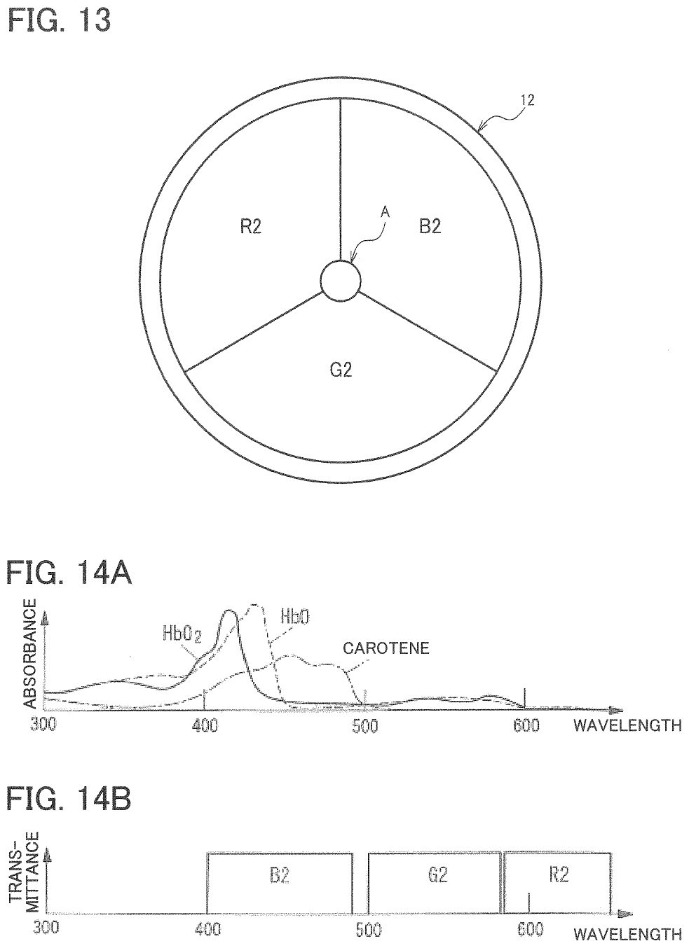

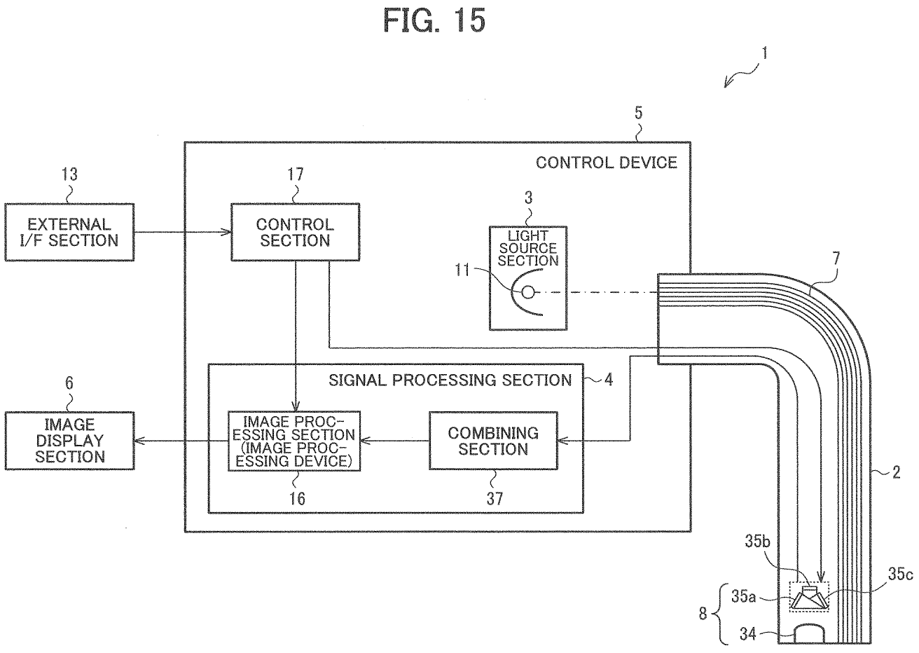

As illustrated in FIG. 13, the filter turret 12 has a filter group that is arranged in a circumferential direction centered on a rotation center A. As illustrated in FIG. 14B, the filter group is formed from filters B2, G2, and R2 that transmit blue light (B2: 400 to 490 nm), green light (G2: 500 to 570 nm), and red light (R2: 590 to 650 nm). As illustrated in FIG. 14A and FIG. 14B, the wavelength band of the filter B2 is a wavelength band in which the absorbances of hemoglobin and carotene are both high. The wavelength band of the filter G2 is a wavelength band in which the absorbances of hemoglobin and carotene are both low. The wavelength band of the filter R2 is a wavelength band in which the absorbances of hemoglobin and carotene are both almost zero.

White light emitted from the xenon lamp 11 passes through the filters B2, G2, and R2 of the rotating filter turret 12 in sequence, and the illumination light of blue B2, green G2, and red R2 are applied to the subject in a time-division manner

The control section 17 synchronizes the timing for capturing by the image sensor 27, the rotation of the filter turret 12, and the timing for image processing by the image processing section 16. The memory 28 stores the image signals acquired by the image sensor 27 in each of the wavelengths of the emitted illumination light. The image processing section 16 combines the image signals in the individual wavelengths stored in the memory 28 to generate a color image.

Specifically, when the illumination light of blue B2 is applied to the subject, the image sensor 27 captures an image and stores the image as a blue image (B channel) in the memory 28. When the illumination light of green G2 is applied to the subject, the image sensor 27 captures an image and stores the image as a green image (G channel) in the memory 28. When the illumination light of red R2 is applied to the subject, the image sensor 27 captures an image and stores the image as a red image (R channel) in the memory 28. Then, when the images corresponding to the illumination light of three colors are acquired, these images are sent from the memory 28 to the image processing section 16. The image processing section 16 performs image processing at the preprocessing section 14 and combines the images corresponding to the illumination light of three colors to acquire one RGB color image. Thus, the image of normal light (white light image) is acquired and output as the captured image to the visibility enhancement section 18.

FIG. 15 illustrates a third modification of the endoscope apparatus according to the present embodiment. Referring to FIG. 15, the so-called 3CCD method is employed. Specifically, a imaging optical system 8 includes a color separation prism 34 that separates reflected light from the subject by wavelength band and three monochrome image sensors 35a, 35b, and 35c that capture light in the individual wavelength bands. The signal processing section 4 includes a combining section 37 and an image processing section 16.

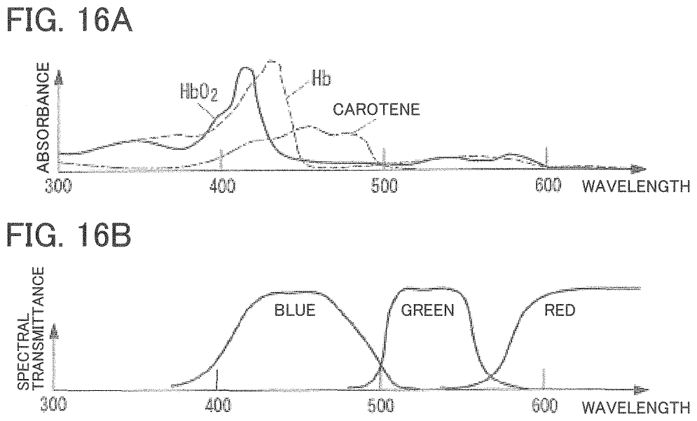

The color separation prism 34 separates the reflected light from the subject into the wavelength bands of blue, green, and red according to transmittance characteristics illustrated in FIG. 16B. FIG. 16A illustrates absorption characteristics of hemoglobin and carotene. The light in the wavelength bands of blue, green, and red separated by the color separation prism 34 respectively enters the monochrome image sensors 35a, 35b, and 35c and is captured as images of blue, green, and red. The combining section 37 combines the three images captured by the monochrome image sensors 35a, 35b, and 35c, and outputs the combined image as an RGB color image to the image processing section 16.

4. Notification Process

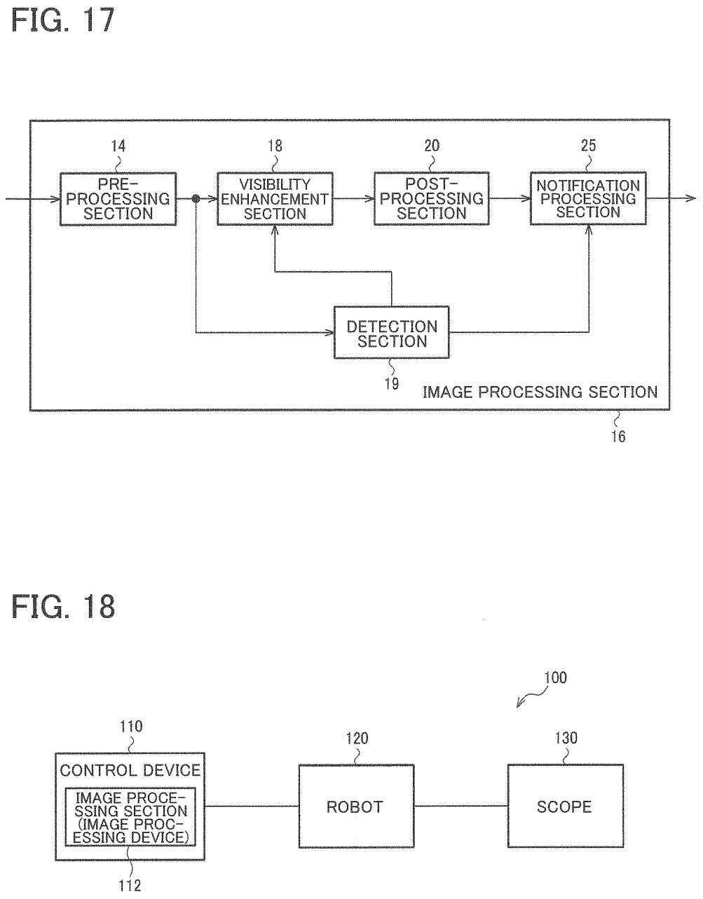

FIG. 17 illustrates a third detailed configuration example of the image processing section. Referring to FIG. 17, the image processing section 16 further includes a notification processing section 25 that performs a notification process based on result of detection of the blood region by the detection section 19. The blood region may be a blood region detected by the blood region detection section 22 illustrated in FIG. 4 (an outflowing blood region in a narrow sense) or may be a blood vessel region detected by the blood vessel region detection section 21 illustrated in FIG. 9.

Specifically, when the detection section 19 detects the blood region, the notification processing section 25 performs the notification process of notifying the user of the detection of the blood region. For example, the notification processing section 25 superimposes an alert indication on a display image and outputs the display image to the image display section 6. For example, the display image includes a region where the captured image is displayed and its peripheral region where the alert indication is displayed. The alert indication is a blinking icon or the like, for example.

Otherwise, the notification processing section 25 performs the notification process of notifying the user that the blood vessel region exists near a treatment tool based on positional relationship information (for example, distance) indicating the positional relationship between the treatment tool and the blood vessel region. The notification process is a process of displaying an alert indication similar to the one described above, for example.

The notification process is not limited to a process of displaying an alert and may be a process of highlighting the blood region (blood vessel region) or a process of displaying characters (text or the like) for attracting attention. Otherwise, the notification process is not limited to notification by image display and may be notification by light, sound, or vibration. In that case, the notification processing section 25 may be provided as a constituent element separate from the image processing section 16. Otherwise, the notification process is not limited to a process of notification to the user and may be a process of notification to a device (for example, a robot in a surgery support system described later). For example, an alert signal may be output to the device.

As described above, the visibility enhancement section 18 suppresses the process of attenuating the colors other than yellow in the blood region (blood vessel region). Therefore, there is a possibility that the chromas of colors of the blood region become low as compared to a case where the process of attenuating the colors other than yellow is not performed. According to the present embodiment, it is possible to, based on the detection result of the blood region (blood vessel region), perform a process of notifying the existence of blood in the captured image or a process of notifying that the treatment tool has approached the blood vessel.

5. Surgery Support System

The endoscope apparatus (endoscope system) according to the present embodiment is assumed to be a type in which a control device is connected to an insertion section (scope) so that the user operates the scope to capture the inside of a body as illustrated in FIG. 2, for example. However, the present disclosure is not limited to this and can be applied to a surgery support system using a robot, for example.

FIG. 18 illustrates a configuration example of a surgery support system. The surgery support system 100 includes a control device 110, a robot 120 (robot main body), and a scope 130 (for example, a rigid scope). The control device 110 is a device that controls the robot 120. Specifically, the user operates an operation section of the control device 110 to move the robot through which to perform surgery on a patient. In addition, the user operates the operation section of the control device 110 to manipulate the scope 130 via the robot 120 and capture a surgical region. The control device 110 includes an image processing section 112 (image processing device) that processes images from the scope 130. The user operates the robot while seeing the images displayed on a display device (not illustrated) by the image processing section 112. The present disclosure can be applied to the image processing section 112 (image processing device) in the surgery support system 100. In addition, the scope 130 and the control device 110 (and also the robot 120) correspond to the endoscope apparatus (endoscope system) including the image processing device according to the present embodiment.

Although the embodiments to which the present disclosure is applied and the modifications thereof have been described in detail above, the present disclosure is not limited to the embodiments and the modifications thereof, and various modifications and variations in components may be made in implementation without departing from the spirit and scope of the present disclosure. The plurality of elements disclosed in the embodiments and the modifications described above may be combined as appropriate to implement the present disclosure in various ways. For example, some of all the elements described in the embodiments and the modifications may be deleted. Furthermore, elements in different embodiments and modifications may be combined as appropriate. Thus, various modifications and applications can be made without departing from the spirit and scope of the present disclosure. Any term cited with a different term having a broader meaning or the same meaning at least once in the specification and the drawings can be replaced by the different term in any place in the specification and the drawings.

What is claimed is:

1. An image processing device comprising a processor including hardware,
the processor being configured to perform:
executing a color attenuation process on a region other than a yellow region in a captured image including a subject image to relatively enhance visibility of the yellow region in the captured image;
detecting a blood region that is a region of blood in the captured image based on color information of the captured image; and
suppressing or stopping the attenuation process on the blood region based on detection result of the blood region.

2. The image processing device as defined in claim 1, wherein
the processor performs
detecting a blood vessel region that is a region of a blood vessel in the captured image based on the color information and structural information of the captured image, and
suppressing or stopping the attenuation process on the blood vessel region based on detection result of the blood vessel region.

3. The image processing device as defined in claim 1, wherein
the processor performs
detecting the blood region based on at least one of the color information and brightness information of the captured image, and
suppressing or stopping the attenuation process on the blood region based on the detection result of the blood region.

4. The image processing device as defined in claim 3, wherein
the processor performs
dividing the captured image into a plurality of local regions and determining whether each of the plurality of local regions is the blood region based on at least one of the color information and the brightness information of the local region.

5. The image processing device as defined in claim 1, wherein
the processor performs
the attenuation process by determining a color signal corresponding to the blood in a pixel or a region of the captured image and multiplying a color signal of the region other than the yellow region by a coefficient varying in value according to a signal value of the color signal.

6. The image processing device as defined in claim 1, wherein
the processor performs
a color conversion process on a pixel value of a pixel in the yellow region so as to rotate toward green in a color space.

7. The image processing device as defined in claim 1, wherein
the color of the yellow region is the color of carotene, bilirubin, or stercobilin.

8. The image processing device as defined in claim 1, wherein
the processor performs
a notification process based on the detection result of the blood region.

9. An endoscope apparatus comprising an image processing device, wherein
the image processing device includes
a processor including hardware,
the processor being configured to perform:
executing a color attenuation process on a region other than a yellow region in a captured image including a subject image to relatively enhance visibility of the yellow region in the captured image;
detecting a blood region that is a region of blood in the captured image based on color information of the captured image; and
suppressing or stopping the attenuation process on the blood region based on detection result of the blood region.

10. The endoscope apparatus as defined in claim 9, comprising
a light source section that emits an illumination light in a wavelength band of normal light.

11. The endoscope apparatus as defined in claim 10, wherein
the light source section
includes one or more light emitting diodes (LEDs), and
emits the normal light by light emission from the one or more light emitting diodes as the illumination light.

12. An operating method of an image processing device, comprising:
executing a color attenuation process on a region other than a yellow region in a captured image including a subject image to relatively enhance visibility of the yellow region in the captured image;
detecting a blood region that is a region of blood in the captured image based on color information of the captured image; and
suppressing or stopping the attenuation process on the blood region based on detection result of the blood region.

13. The operating method of an image processing device as defined in claim 12, comprising:
detecting a blood vessel region that is a region of a blood vessel in the captured image based on the color information and structural information of the captured image; and
suppressing or stopping the attenuation process on the blood vessel region based on detection result of the blood vessel region.

14. The operating method of an image processing device as defined in claim 12, comprising:
detecting the blood region based on at least one of the color information and brightness information of the captured image; and
suppressing or stopping the attenuation process on the blood region based on the detection result of the blood region.

15. The operating method of an image processing device as defined in claim 14, comprising
dividing the captured image into a plurality of local regions and determining whether each of the plurality of local regions is the blood region based on at least one of the color information and the brightness information of the local region.

16. The operating method of an image processing device as defined in claim 12, comprising
executing the attenuation process by determining a color signal corresponding to the blood in a pixel or a region of the captured image and multiplying a color signal of the region other than the yellow region by a coefficient varying in value according to a signal value of the color signal.

17. The operating method of an image processing device as defined in claim 12, comprising
performing a color conversion process on a pixel value of a pixel in the yellow region so as to rotate toward green in a color space.

18. The operating method of an image processing device as defined in claim 12, wherein
the color of the yellow region is the color of carotene, bilirubin, or stercobilin.

19. The operating method of an image processing device as defined in claim 12, comprising
performing a notification process based on the detection result of the blood region.
